(19) United States
(12) Patent Application Publication
(10) Pub. No.: US 2012/0059041 A1
(43) Pub. Date: Mar. 8, 2012

(54) Methods for Extending Lifespan in Subject

(75) Inventors: Horng-Dar WANG, Hsinchu (TW); Chiou-Hwa YUH, Hsinchu (TW); Wan-Chih LU, Hsinchu (TW)

(73) Assignee: NATIONAL TSING HUA UNIVERSITY, Hsinchu (TW)

(21) Appl. No.: 12/633,640

(22) Filed: Dec. 8, 2009

(30) Foreign Application Priority Data
Aug. 24, 2009 (TW) 098128405

(51) Int. Cl. A61K 31/7105; C07H 21/04; A61P 39/00; A61K 31/7088

(52) U.S. Cl. 514/44 A; 514/44 R; 536/23.1; 536/24.5

(57) ABSTRACT

Disclosed is a method for extending lifespan in a subject. By screening for mutations that enhance resistance to multiple stresses, the invention identified multiple alleles of alpha-1, 2-mannosidase I (mas1) which, in addition to promoting stress resistance, also extended longevity. Meanwhile, longevity enhancement of a subject is also observed when either the expression of mas1 or its downstream gene Edm1 is reduced. Furthermore, this invention also found that the down-regulating mas1 and Edm1 may extend longevity by modulating DR (Dietary Restriction). Thus, via molecular biology techniques, the expression of the target genes such as mas1 and Edm1 can be regulated, and the lifespan extension for a subject also can be achieved.

### FIELD OF THE INVENTION

The present invention relates to the field of anti-aging, and in particular to a method for extending lifespan in a subject.

### BACKGROUND OF THE INVENTION

Aging is a complicated process influenced by numerous genetic and environmental factors. Several mechanisms have been proposed to regulate aging, including the accumulation of damage resulting from reactive oxygen species, the loss of genomic integrity, as well as the modulation of genetic pathways that control reproductive output, the ability to withstand environmental stress, and nutrient utilization. During the natural aging process, the increased expression of stress-responsive genes is often observed and in many cases, long-lived individuals display increased resistance to environmental stressors. Thus, exposure to proper levels of stress in an individual may results in enhanced longevity.

In previous studies, a common stressor may extend longevity is dietary restriction (DR). The expression of endoplasmic reticulum (ER) stress response related genes, such as Bip/Grp78, may be down-regulated under this condition. Bip/Grp78 (immunoglobulin binding protein/glucose regulated protein 78), is a molecular chaperone that uses ATP/ADP cycling to regulate protein folding by the protein disulfide isomerase (PDI) family of proteins. It is a 78 kDa glucose-regulated heat shock protein and is involved in unfolded protein response. Bip/Grp78 is highly conserved protein that is essential for protein folding, ER calcium binding, and controlling of the activation of transmembrane ER stress sensors. The promoter of Bip/Grp78 contains cis regulatory elements such as ER stress response element (ERSE) and cAMP response element (CRE), thus activating transcription factor 6 (ATF6) may bind to these element and regulate Bip/Grp78 transcription.

Mas1 is expressed in the ER, the Golgi apparatus, and the lysosome. It is a member of the class I glycosidases and is involved in N-linked glycosylation (Herscovics, 2001). During the calnexin/calreticulin cycle, Mas1 removes mannose from permanently unfolded proteins, then the de-mannosed proteins are recognized by ER degradation-enhancing alpha-1,2-mannosidase-like protein (Edem), and degraded by ER-associated degradation (ERAD). Several lines of evidence indicate that Mas1 is important during the aging process. First, altered N-linked glycosylation affects the maturation rate of proteins that influence longevity, such as insulin and insulin-like growth factor-I receptors. Furthermore, the expression of mas1 is decreased in aging and oxidatively-stressed Drosophila.

Thus, it is desired to identify genes involved in lifespan extension and thus offer a new possible target for anti-aging study.

### SUMMARY OF THE INVENTION

Thus, an objective of the present invention is to provide a method for extending lifespan in a subject. The method comprises the step of altering the protein expression level of at least one multiple stress activating protein to extend the lifespan in the subject, wherein the multiple stress activating protein is selected from the group consisting of alpha-1,2-mannosidase I (mas1) and ER degradation-enhancing alpha-1,2-mannosidase-like protein (Edm1).

Another objective of the present invention is to a DNA fragment for regulating mas1 gene expression, wherein the DNA fragment has a nucleotide sequence of SEQ ID NO: 1. The RNA transcript transcripted from the DNA fragment can bind to the 3′ UTR of mRNA from mas1, so as to down-regulate mas1 expression.

According to the present invention, by screening a series of mutant flies under multiple stress conditions, one of the mutant lines, EP1130, displayed lifespan extension. One transcript from the region adjacent to mutation site, with a length of 1.6-Kb, was found to be differentially expressed in EP1130. A 480-bp sequence at the 3′ end of p1130 is complementary to the 3′ UTR of mas1. This transcript may bind to mRNA of mas1, and down-regulate the expression of mas1

Through molecular biotechnology, Mas1 and its downstream gene Edm1 are mutated to generate mutant strains. It is found that the lifespan of the mutants can be extended. Moreover, double mutants in these two genes show produce no additional enhancement. It appears that Mas1 and Edm1 function in the same pathway. The present invention also depicts that mutation in mas1 may regulate the expression of ER stress related genes such as Bip. is thus down-regulated because of mas1 mutation. It is known that Bip gene is related with dietary restriction (DR) stress regulation. The present invention discloses that reduced marl expression can extend lifespan in an individual under DR stress. The aforementioned biotechnology comprises inserting transposon into target genes, including mas1 and Edm1 gene. Moreover, RNA interference (RNAi) is also used to silence the expression of mas1 gene. The method comprises the steps of: A selected vector is used to express the double-stranded RNA of specific sequence. The dsRNA is reverse transcripted from mRNA of mas1, amplified by PCR primers in an inverted orientation cloned into the vector. The construct is verified by DNA sequencing before micro-injecting to the subject. Depending on the species, a da-Gal4(ubiquitous Gal4 driver) may used to cross with the mutant to express dsRNA to silence mas1 expression.

Thus, the present invention can be practiced in a simple process to extend lifespan in a subject.

### BRIEF DESCRIPTION OF THE DRAWINGS

The present invention will be apparent to those skilled in the art by reading the following description of preferred embodiments thereof, with reference to the attached drawings, in which:

FIG. 1 The survival curves of mutant flies compared to the control. EP1130, EP982 and EP1628 mutants are inserted with the transposable element at mas1 gene. Lifespan of EP1130, EP982 and EP1628 mutants are enhanced, compared to the control w¹¹¹⁸, under oxidative stress caused by little amount of paraquat (oxidant).

FIG. 2 The survival curves of (A) Male mas1 mutants and (B) virgin female mutants relative to the control, respectively. EP1130, EP982 and EP1628 are mutants inserted with the transposable element at mas1 gene. Lifespan of EP1130, EP982 and EP1628 mutants are enhanced, compared to the control w¹¹¹⁸, under oxidative stress caused by little amount of paraquat (oxidant).

FIG. 3 The mRNA expression of mas1 gene measured by Real-time PCR. Relative to the control strain w¹¹¹⁸, mas1 expression of EP1130, EP982 and EP1628 mutants are decreased.

FIG. 4 C. elegans fed with E. coli expressing double-stranded RNA against D2030.1. It is shown that knockdown of D2030.1 expression extends lifespan compared to the control.

FIG. 5 The graph of survival curves of (A) male Edem1 mutant and (B) virgin female Edem1 mutant relative to the control, respectively. Edem1 mutant EP1588 exhibits extended lifespan compared to the control w¹¹¹⁸.

FIG. 6 Dietary restriction treatment to (A) mas1 mutants EP1628, EP982 and (B) Edm1 mutant EP1588. Lowered Bip expression is observed in EP1628, EP982 and EP1588 compared to the control W¹¹¹⁸, measured by RT-PCR, and rp49 is used as an internal control.

FIG. 7 With dietary restriction treatment, mutants EP1130, EP1628, EP982 and EP1588 exhibit extended lifespan relative to the control W¹¹¹⁸.

FIG. 8 The older mice exhibit lower mas1 expression in mice experiment. Mas1 expression of 3-month, 21-month and 28-month old mice are measured by Q-PCR.

### DETAILED DESCRIPTION

Embodiments of methods for extending lifespan in a subject are described herein. In the following description, numerous specific details are described to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail but are nonetheless encompassed within the scope of the invention.

### Embodiment 1

By screening a series of mutant flies under multiple stress conditions, one of the mutant lines, EP1130, displayed lifespan extension. One transcript from the region adjacent to mutation site, with a length of 1.6-Kb, is found to be differentially expressed in EP1130. A 480-bp sequence at the 3′ end of p1130 is complementary to the 3′ UTR of mas1. This transcript may bind to mRNA of mas1, and down-regulate the expression of mas1. The expression of p1130, mas1 mRNA and CG12643 (as an internal control) are measured by RT-PCR. The results reveal that decreased expression of mas1 is observed relative to control w¹¹¹⁸. However, reduced mas1 expression in EP1130 is also observed.

### Embodiment 2

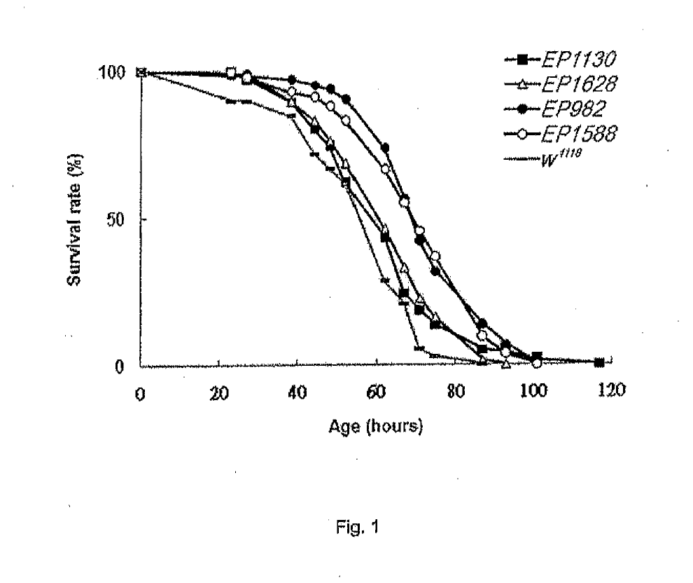

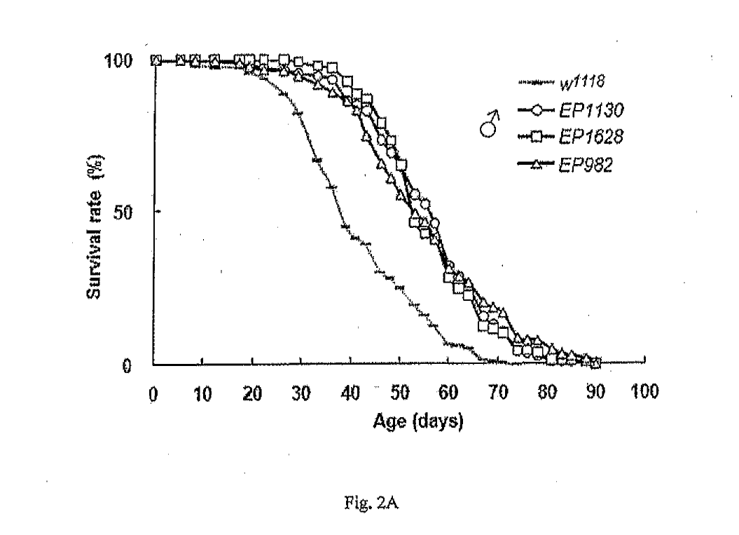

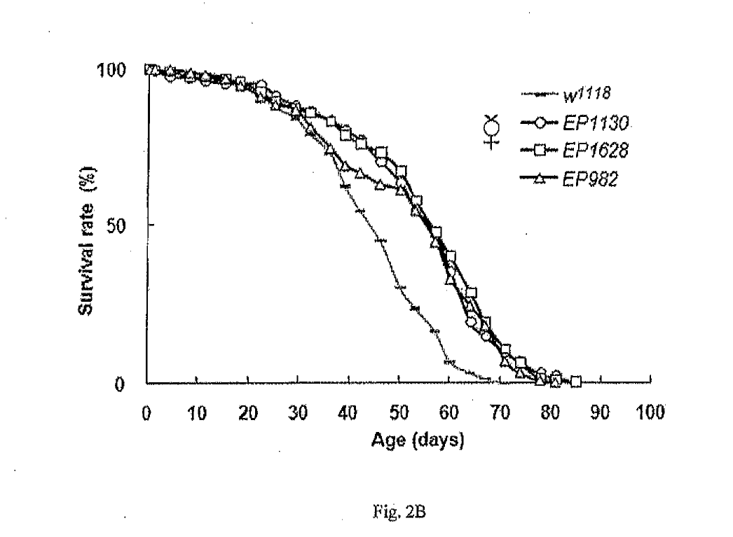

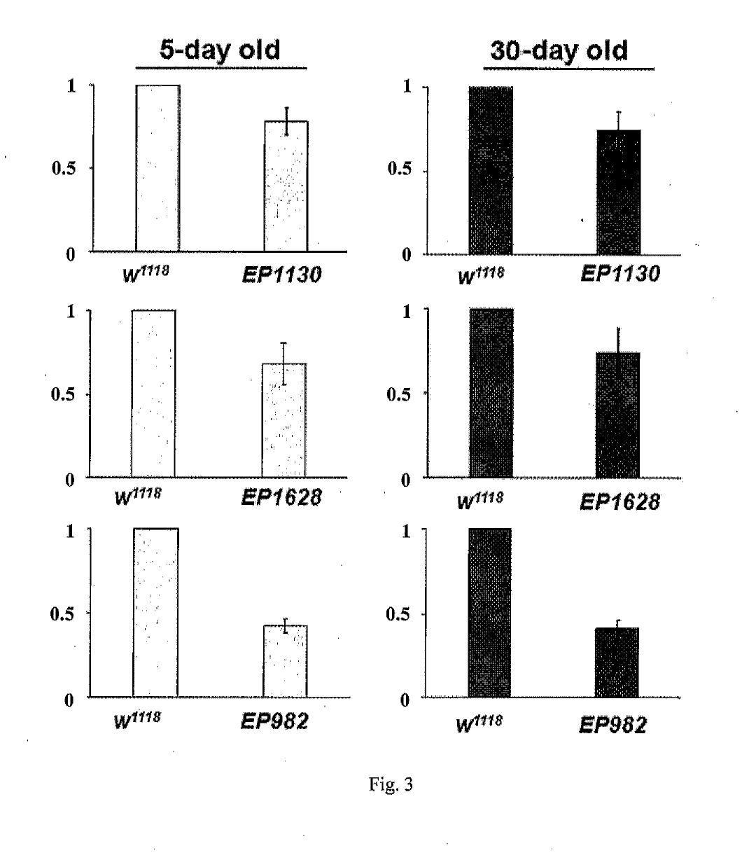

Drosophila melanogaster is used as experimental material. To generate mutants, transposon is inserted into mas1 of flies. The transposon-mediated mutants include EP1130, EP982 and EP1628 (purchased from Bloomington Drosophila Stock Center). In addition, two environmental stress, paraquat and starvation, are applied to screen mutants. Under this condition, EP1130, EP982 and EP1628 display a similar level life extension, 38%, 36% and 39%, respectively. The survival curve of male mutant is shown as FIG. 1. Furthermore, male mutants and virgin female mutants also exhibit enhanced lifespan compared to the control (as shown in FIG. 2(A), 2(B)). To confirm that the longevity changes are due to the down-regulation of mas1, the level of expression of this gene is examined in EP1130, EP982 and EP1628 by real time PCR. It is found that the expression of mas1 is reduced in young and old flies in all three mutants, compared to the control w¹¹¹⁸. (as shown in FIG. 3) These results suggest that mutant with insertion into mas1 may exhibit reduced mas1 expression. Thus the mutant shows enhanced environmental stress resistance and extends its lifespan.

### Embodiment 3

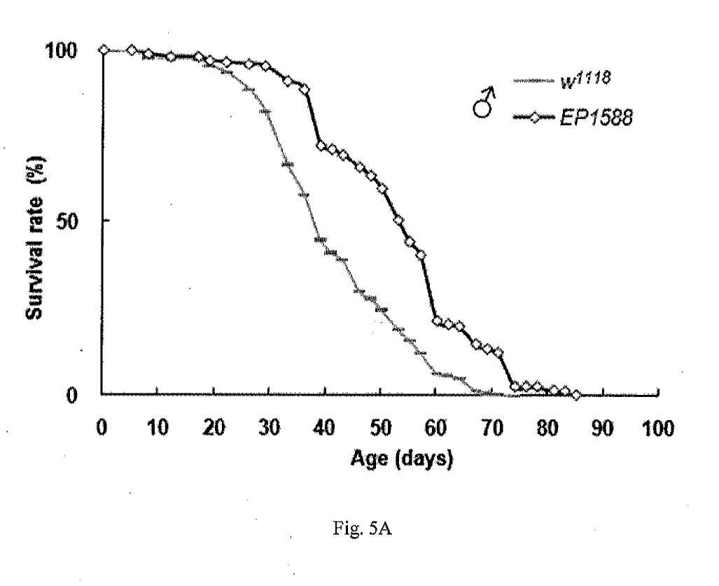

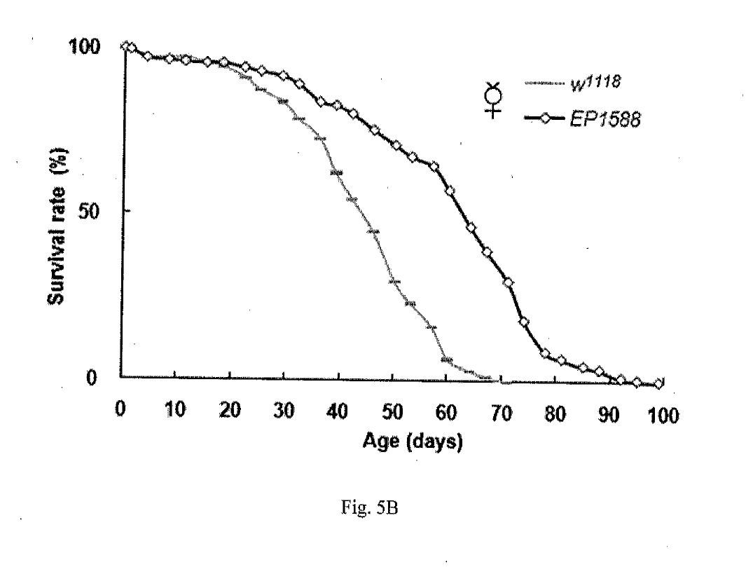

Drosophila melanogaster is used as experimental material. Similarly, Edm1, a gene downstream of mas1, is inserted with transposon to generate mutant EP1588. The expression level of Edm1 is significantly lower in the mutant relative to the control. In addition, the mean lifespan of both male and female mutant flies is increased by more than 30%. (as shown in FIG. 5(A)˜5(B)) Edm1 mutant EP1588 exhibits extended lifespan compared to the control w¹¹¹⁸. Furthermore, EP1588, EP1130, EP982 are crossed to EP1628, and the result shows that similar lifespan extension without synergistic effect is obtained. Therefore, it is proved that two genes function in the same pathway (as shown in Table. 1).

TABLE 1

| Strain [symbol] | Sample size | Mean lifespan | Increase | P -value |
|---|---|---|---|---|
| w¹¹¹⁸ | 153 | 44 | | |
| EP1130/+ | 62 | 65 | 48% | <0.0001 |
| EP1628/+ | 166 | 57 | 30% | <0.0001 |
| EP982/+ | 100 | 57 | 29% | <0.0001 |
| EP1588/+ | 220 | 52 | 19% | <0.0001 |
| EP1130/1628 | 148 | 57 | 28% | <0.0001 |
| EP1130/982 | 83 | 56 | 27% | <0.0001 |
| EP1628/982 | 232 | 54 | 23% | <0.0001 |
| EP1588/1130 | 221 | 58 | 32% | <0.0001 |
| EP1588/1628 | 219 | 62 | 40% | <0.0001 |
| EP1588/982 | 208 | 60 | 36% | <0.0001 |

P-value is calculated by log-rank test.

### Embodiment 4

A method to generate a double-stranded RNA expressed mutant flies, comprises the following steps: (a) The pWIZ vector is used to express the double-stranded RNA of a 220-bp sequence. The 220-bp dsRNA is from CG32684 cDNA (reverse transcripted from mRNA of mas1) amplified by PCR primers (MA220 FOR and MA220 REV) in an inverted orientation cloned into pWIZ. (b) The construct is verified by DNA sequencing before micro the micro-injection to generate the RNAi transgenic flies, which is capable of expressing the dsRNA.

A da-Gal4(ubiquitous Gal4 driver) is used to cross with the mutant to express dsRNA to silence the target gene mas1.

The results show that lifespan of mutant flies extend 39% greater than that of control flies. It is proved that lifespan of flies can be extended through lowering magi expression via RNAi technology.

### Embodiment 5

A method to generate a double-stranded RNA expressed mutant fly, comprises the following steps: (a) E. coli containing the construct expressing dsRNA against D2030.1 (mas1 orthologue in worm) is provided. (b) Feeding worms (C. elegans) with the E. coli of step (A). The worms fed with vector alone are used as control.

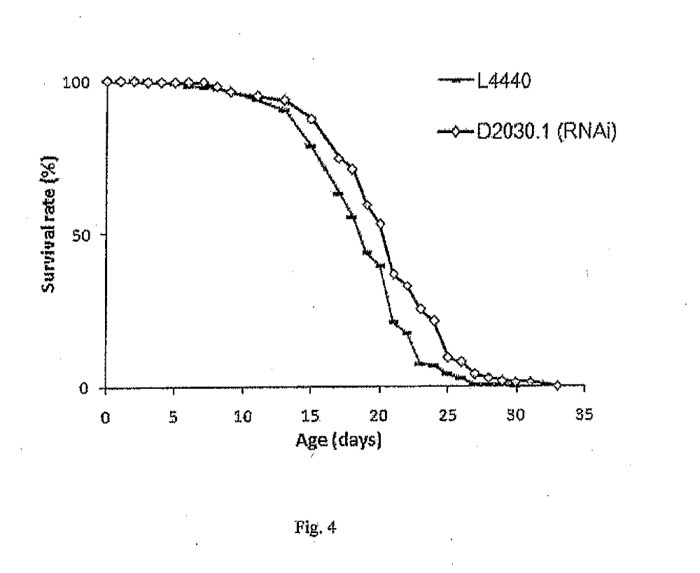

The results show that lifespan of mutant worms extend 9% greater than that of control worms. As in the fly, it is proved that lifespan of worms can be extended through lowering mas1 expression via RNAi technology (as shown in FIG. 4).

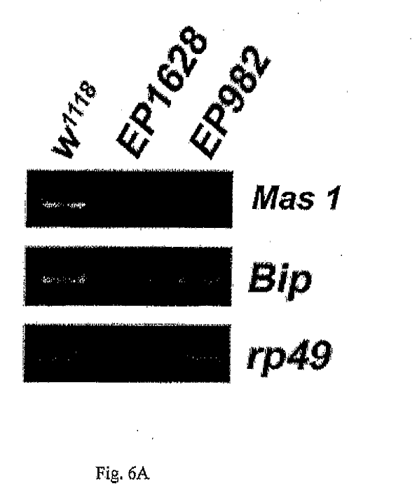

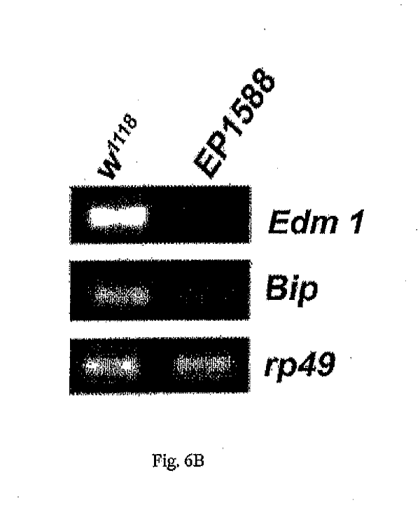

Refer to FIG. 6(A)˜6(B), it is shown that the mRNA expression of Bip/Grp78, a dietary restriction stress regulation related gene, with dietary treatment, compared to the control w¹¹¹⁸. The results reveal that Bip expression of EP1130, EP1628, EP982 and EP1588 are reduced. Moreover, in both Drosophila and C. elegans, Bip expression is reduced in mas1 knockdown flies and in D2030.1 knockdown worms, respectively. Under abundant or restricted food condition, mutant flies exhibit longer lifespan under both conditions. It appears that the lifespan modulation of mutant is similar to that under dietary restriction condition, which is associated with down-regulation of mas1 or Edm1.

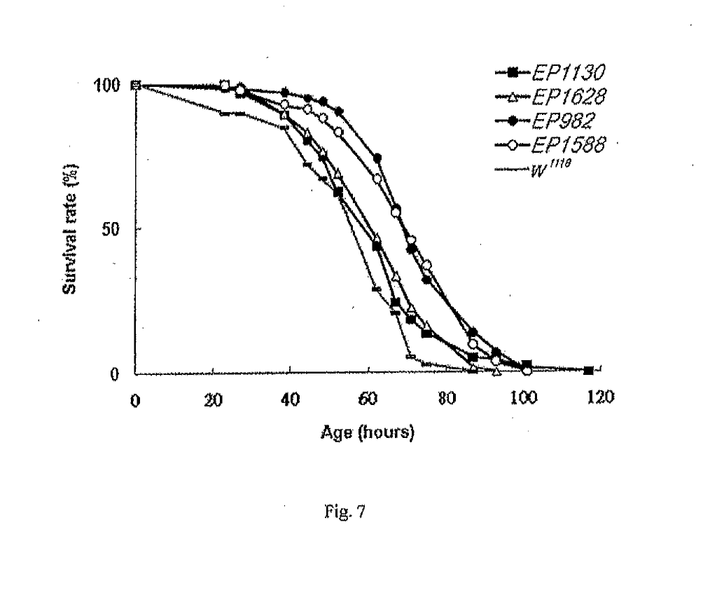

Refer to FIG. 7, it is shown that mutants EP1130, EP1628, EP982 and EP1588, with dietary restriction treatment, exhibit extended lifespan relative to the control w¹¹¹⁸, respectively. These results show that down-regulation of mas1 or Edm1 can extend lifespan in a subject, as under dietary restrict environment. Additionally, better stress resistance and longer lifespan are also performed.

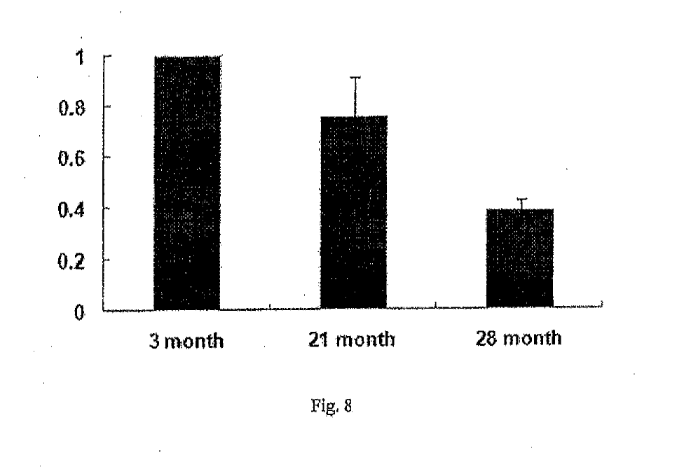

Refer to FIG. 8, in mice experiment, mas1 expression of 3-month, 21-month and 28-month old mice are measured by Q-PCR. The result shows that the older mice exhibit lower mas1 expression.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A method for extending lifespan in a subject, comprising the step of altering the protein expression level of at least one multiple stress activating protein to extend the lifespan in the subject, wherein the multiple stress activating protein is selected from the group consisting of alpha-1,2-mannosidase I (mas1) and ER degradation-enhancing alpha-1,2-mannosidase-like protein (Edm1).

2. The method as claimed in claim 1, wherein the step of altering the protein expression level includes reducing the multiple stress protein expression level, mutating or removing the multiple stress protein.

3. The method as claimed in claim 1, wherein the multiple stress protein further comprises immunoglobin binding protein (BiP/GRP78).

4. The method as claimed in claim 2, wherein the means of altering the protein expression level includes microinjection, transposon insertion, RNA-interference, antisense technology, or gene knockout.

5. The method as claimed in claim 1, wherein the subject is under multiple stress conditions.

6. The method as claimed in claim 1, wherein the multiple stress conditions further include treating the subject with a dietary restriction.

7. The method as claimed in claim 1, wherein the multiple stress conditions further include treating the subject with a determined amount of oxidant.

8. The method as claimed in claim 1, wherein the multiple stress conditions further include treating the subject with adequate dietary.

9. The method as claimed in claim 1, wherein the subject is an insect.

10. The method as claimed in claim 1, wherein the subject is a protostome.

11. The method as claimed in claim 1, wherein the subject is a mammal.

12. The method as claimed in claim 1, wherein the subject is a human.

13. A DNA fragment for regulating mas1 gene expression, having a nucleotide sequence of SEQ ID NO: 1.

14. The DNA fragment as claimed in claim 13, wherein RNA transcript transcripted from the DNA fragment can bind to the 3′ UTR of mRNA from mas1, so as to downregulate mas1 gene expression.


SEQUENCE LISTING (1 sequence)

SEQ ID NO: 1 - DNA, 1523 bp, Drosophila melanogaster
tggtctggtctggcgggctcgtctagtctgctcgacaaagaagagcgaatgagagggaaaacgaccggtctgatatggtacactaaaagaaacgtcaagctactttctgatttatatgccgaaaaatcacttttaatgcgaggtggccactggcatggtaaactatgtcggcaaagtcaaagctacagttttaaatccataaaatggtaaaatctttccctctgagagagatcctgttatatttctttgggtgtactctctcgcatgcttgttctcgcatttttttggaagtgggcggtgggtggcgaatgcaatcgccagttggcactcctccattttatcgttatgctttgttcgcattcgattcagcgttttactgccccttcttctgaggatcttatgctggcaaaatttggactttgcacgacaatcggatgttaacccattacgttaaaaaatccattttaaatgtacatatatgtatatctatatttagaaaactctttcccaaagcaatatattttttaagcttcgaatttttgatatgtatctacatatatctaaagagtatttcttgaacaatcatgattcaaggggttaatcgaagtggctggaagggtggatatgtcgcgacgaactccctttccgggttctcgaatcgccagcgaatgttgttaacctcacattgtgctttgtgtgttcgccttttgtgcaagtttcttcattaagtatacgcacattcggtttaagtggactgctcattaattgagctcgacaactgctcgtgaagtggcttgtgtggcagatacataaatatatctgaaagtggaatcagcgggttatgggggggcactagtttatagtctgacaaactatcaatgagctatcaatgtgctgcgagataaaaatgtttttacaaagaaagagctgaataagcggtgaattttccaacattacgtatgagagtataataaaatatgcatattttcaaaccttttttaaagcatttgagtaccgtaagaatgaaataaataacgcacagttataagttaattattagatctttattaaacttatgatttaggcaaaatacacgtgacatctccagcagctcagggactgctattgatgaatgtattgtgtctgatgcatttacaaactgccattcgaatctgtgtgttcggcattataatcggatatattatatatctcgctattggcctcaatttctaatttgatctaaacaaaaaaaggtttgctctagagactaaatttatgcttaattagtatcgaattcattcttttcttcctattctttttaagcatttagctcgtttaacgtttgttagcttgggtttattagatttcttcatttatcgggttaaatcattttgatattattttattttgttaaaatgtttctttaaaattcttttgttttacattagggtcttagcttatgttttatgttatttttacgtttgttaaatcactttacacagaacaagctgtgtatgttacaa